(19) United States
(12) Patent Application Publication
(10) Pub. No.: US 2026/0229367 A1
(43) Pub. Date: Aug. 6, 2026

(54) STRUCTURED DATA CAPTURE, AGGREGATION, AND ANALYSIS OF CLINICAL CHARACTERISTICS OF PATIENTS IN A LIVER DISEASE DIAGNOSTIC SETTING

(71) Applicants: Roche Molecular Systems, Inc., Pleasanton, CA (US); Roche Diagnostics International AG, Rotkreuz (CH)

(72) Inventors: Supretta Yadav BARIYA SHANKER, Pleasanton, CA (US); Ravinder CHANA, Rotkreuz (CH); Harish HEBBAR, Pleasanton, CA (US); Ritika NARULA, Pleasanton, CA (US); Ravinder PABIAL, Rotkreuz (CH); Mahesh PALAN, Pleasanton, CA (US); Manan Mahesh PANCHOLI, Pleasanton, CA (US); Kaushal Dilip PAREKH, Pleasanton, CA (US)

(21) Appl. No.: 19/635,390

(22) Filed: Mar. 31, 2026

Related U.S. Application Data
(63) Continuation of application No. PCT/US2024/049241, filed on Sep. 30, 2024.
(60) Provisional application No. 63/587,272, filed on Oct. 2, 2023.

(51) Int. Cl. G16H 50/30; G16H 10/60; G16H 20/00; G16H 50/70

(52) U.S. Cl. CPC G16H 50/30; G16H 10/60; G16H 20/00; G16H 50/70

(57) ABSTRACT

A system for managing liver disease including an electronic medical record system comprising a plurality of patient records. The system includes a patient management platform configured to access the electronic medical record (EMR) system to retrieve medical records of one or more patients, analyze the medical records to selectively identify data relating to liver condition(s), interface with one or more data analytics platform(s), the data analytics platform(s) configured to receive and analyze the data relating to liver-conditions. Based on the analyzing by the patient management interface and/or the data analytics platform(s), generating a display including specific liver-related patient attributes and conditions, and at least one of a liver-health prognosis or recommended treatment for addressing the liver condition(s) respective to the one or more patients. The liver conditions may include one or more of: cancer, cirrhosis, liver failure, fatty liver disease, steatosis, ischemia, and/or hepatitis.

### CROSS-REFERENCE TO RELATED APPLICATIONS

This application is a US bypass continuation of International Application No. PCT/US2024/049241, filed on Sep. 30, 2024, which claims the benefit of and priority to U.S. Provisional Application No. 63/587,272, filed on Oct. 2, 2023, the entire disclosures of which are hereby incorporated herein by reference in their entireties for all purposes.

### BACKGROUND

Liver disease comes in many complex forms with overlapping combinations of symptoms and targeted treatments for specifically addressing them. Monitoring, identifying, and managing the sources of symptoms and providing an accurate diagnosis and treatment plan can therefore become a difficult task. Often the pertinent medical information (e.g., blood tests, liver scans, pathology reports, etc.) can come from disparate sources in varying formats. Combining the information in a patient's record and placing it into context for proper review by medical professionals may therefore become time consuming and expensive.

### BRIEF SUMMARY

Systems and methods for managing liver disease are described, which may include a patient management platform connected with and configured to access an electronic medical record (EMR) system of patient medical records. The platform is configured to analyze medical records to selectively identify data pertaining to liver conditions, analyze the pertinent data, and to generate a display and/or a report including specific liver-related patient attributes and conditions based on the analysis. Treatment(s) for the conditions may also be identified and presented. In some embodiments, the conditions may include one or more of cancer, cirrhosis, liver failure, fatty liver disease, steatosis, ischemia, and/or hepatitis.

The system may be accessible such as by use of an application programming interface (API) accessible from or with other medical data systems (e.g., EPIC). Data may be ingested or communicated such as through a standardized interface (e.g., Fast Healthcare Interoperability Resources (FHIR)). Data processing, including data storage and analysis, may be performed with the use of cloud services (e.g., AWS, GCP) in order to improve efficiency and security.

In some embodiments, the system is configured to receive unstructured or structured data including information relating to liver disease, structuring the data into a predefined format identifying data pertaining to liver conditions, and storing the structured data in the predefined format within records of the EMR. The structuring may be performed such as by use of OCR, natural language processing (NLP), and/or machine learning models. The predefined format may include a common schema or ontology of liver-related terminology.

In some embodiments, modules particular to particular types of data, including laboratory data and biomarkers (e.g., based on pathology, genomics, and imaging), are configured to perform processing/analysis of the data types independently and/or in concert. For example, a biomarker module may be configured to identify particular genetic mutations relating to liver disease in combination with imaging data analysis to make an overall prognosis (e.g., utilizing machine learning).

In some embodiments, independent or external analytics tools may be accessed, such as through its own and external APIs, in order to complement analytics and identification/treatment of liver conditions. In some embodiments, the system can be interfaced with external collaborators and/or data sources that may include clinical data (e,g, clinical trials data) or generalized information about liver conditions and treatment (e.g., treatment guidelines). In some embodiments, the access is configured to anonymize patient data transmitted to external tools. Such clinical data may be used, for example, as training data for machine learning models. Updated information on treatment guidelines or clinical data may be utilized to improve diagnosis, prognosis, clinical decisions, and/or treatment of patients.

### BRIEF DESCRIPTION OF THE DRAWINGS

The detailed description is set forth with reference to the accompanying figures.

FIG. 1 is an illustrative diagram of a system for managing liver disease according to some embodiments.

FIG. 2 is an illustrative diagram of a system for managing liver disease according to some embodiments.

FIG. 3 is an illustrative diagram of a data model for a liver disease management system according to some embodiments.

FIG. 4 is an illustrative diagram of a user interface for structuring data in a liver disease management system according to some embodiments.

FIG. 5A is an illustrative diagram of a user interface for structuring and reporting data and analysis in a liver disease management system according to some embodiments.

FIG. 5B is an illustrative diagram of a user interface for structuring and reporting imaging data and analysis in a liver disease management system according to some embodiments.

FIG. 6 is an illustrative diagram of a process of structuring and transforming medical data in a liver disease management system according to some embodiments.

FIG. 7 is an illustrative process flow diagram for generating and reporting liver disease data and clinical analytics according to some embodiments.

FIG. 8 illustrates an example computer system that may be utilized to implement techniques disclosed herein.

### DETAILED DESCRIPTION

Disclosed herein are systems and methods for liver disease management. A system includes a medical data management and analytics platform. The platform may include data integration components configured to incorporate medical data from disparate sources, including electronic medical record (EMR) systems, biomarkers, diagnostic systems (e.g., imaging, pathology, genomics), clinical data resources (e.g., clinical trials data), and data collection interfaces (e.g., patient reports, remote patient monitoring), and identify/collate data particular to liver conditions and diseases.

A liver disease management system may include an analytics platform (e.g., machine learning) for utilizing identified/collated liver-related data for providing clinical decision support (e.g., diagnosis, prognosis, suggested clinical decisions). In some embodiments, the system is configured to interface with external tools (e.g., analytical tools) or platforms that provide support for making clinical decisions or for collaboration (e.g., research institutions). This interface may be compatible with particular standardized interfaces (e.g., FHIR) and underlying data may be managed through a cloud computing/data platform.

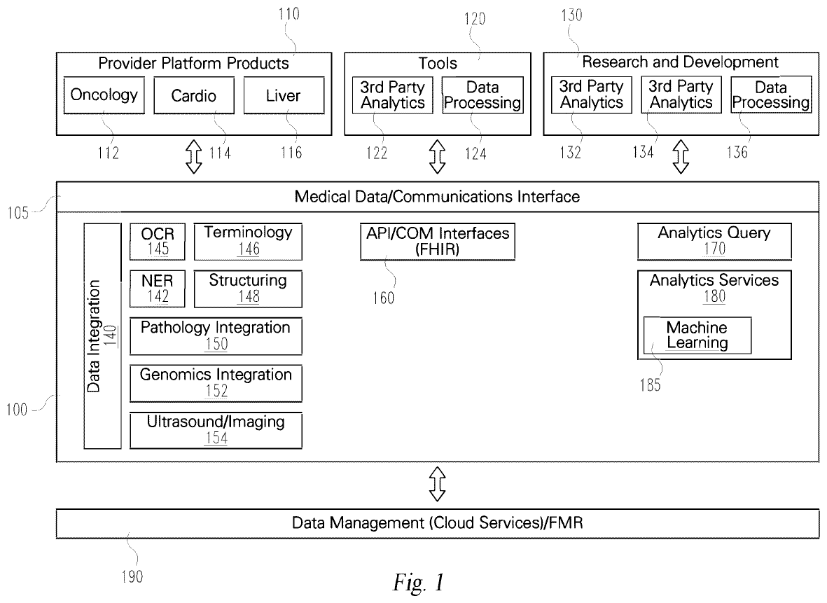

FIG. 1 is an illustrative diagram of a system for managing liver disease according to some embodiments. A base data processing/management system or platform 100 for managing liver disease includes multiple modules for managing medical data and analytics. A data integration interface 140 is configured to receive structured or unstructured medical data and structure the data according to a predetermined particular form if applicable for identification and analytics operations pertaining to liver disease. Data not already in a digital format may be scanned utilizing an OCR module 145, and data that is not formatted according with particular terminology and structure may be transformed in such a format utilizing a named entity resolution (NER) module 142, terminology module 146, and structuring module 148. For example, certain liver conditions may be identified within a record or report utilizing the NER module 142 and mapped to particular terminology or codes 146 and structured into a predefined form utilizing structuring module 148 (e.g., a database record with predetermined fields). Predefined formats may include, for example, SNOMED-CT, LOINC, International Classification of Diseases (ICD), and/or United Code for Units of Measure (UCUM) Data of particular types (e.g., pathology data, genomics data, and/or imaging data) may be transformed utilizing specialized modules (pathology integration module 150, genomics integration module 152, and imaging module 154). For example, radiology (e.g., X-RAY, MRI, PET) and ultrasound images may be transformed images into a particular imaging format (e.g., DICOM, UFF) and according to particular reporting structure (e.g., for reporting a liver condition). Data pertaining particularly to liver conditions can be selectively identified as such in a data storage system (e.g., a data management module 190). Data management module 190 can be a cloud based platform, for example, and/or utilize internal or external electronic medical record (EMR) systems.

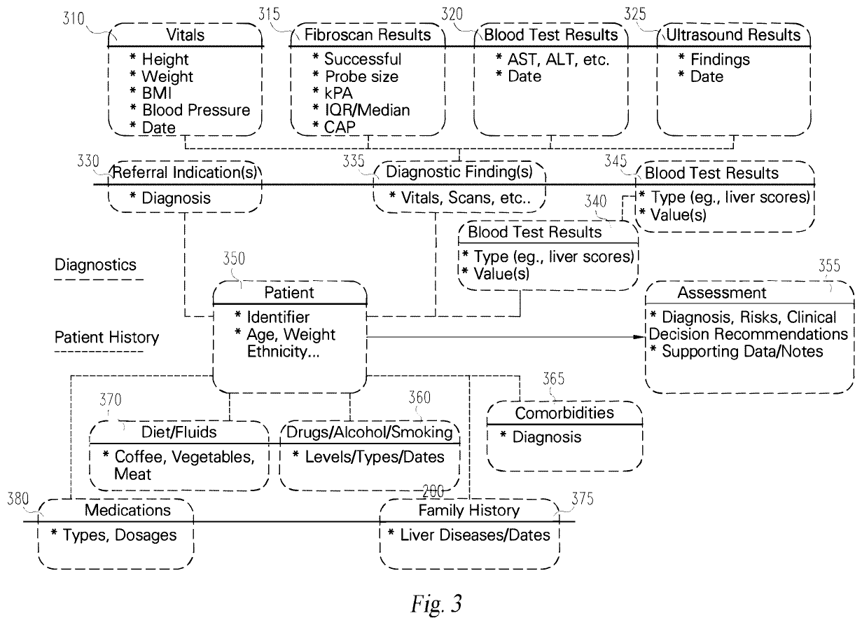

Data processing/management system and platform 100 includes an analytics services module 180 configured to perform analytics on medical data such as for predicting/identifying medical conditions/outcomes, identifying biomarkers, and suggesting clinical steps (e.g., treatments). The services may include machine learning module 185 that can be trained with medical data from patients and/or from clinical data. An analytics query module 170 provides an interface for submitting queries to the analytics services module 180. Queries may include structured or unstructured (e.g., chat style) queries for analytical results or predictions pertaining to liver conditions (e.g., a clinical trial that may match with a patient's condition, likelihood of success for a particular treatment, expected survival rate for a patient, and other queries). For example, a query interface can be configured to prompt a user for patient characteristics and/or histories (e.g., as illustrated in FIG. 3) to identify risks or likelihood for liver conditions and/or recommend treatments/lifestyle changes.

Data processing/management system 100 includes a communications interface 105 for communicating with provider-facing platforms (e.g., a liver disease-specific front-end interface) and external users and systems (e.g., medical providers, third party services, and collaborative institutions). A provider platform 110 includes interfaces/platforms 112, 114, and 116 particular to managing oncology, cardiology, and liver-related conditions, for example. The interface may be configured with or configured to utilize an API and protocol for such communications. The protocol/API may utilize a standardized format/structure such as the Fast Healthcare Interoperability Resources (FHIR) standard, for example.

Data processing/management system 100 may utilize or share data from/with external sources or collaborators 130. Such sources can include clinical trial data 132 such as from trials of treatments for liver disease. Data from patients can also be utilized with drug-discovery platforms 134 such as for identifying biomarkers and/or identifying potential therapeutics for treating liver conditions. Data may be shared with or obtained from collaborators 136 (e.g., academic research institutions). Data from such sources can be utilized with the analytics services module 180. In some embodiments, data includes clinical data and/or standard of care guidelines (e.g., American Association for the Study of Liver Diseases (ASLD) Guidelines) that may be accessible such as at a clinical trials repository or other data source.

Third party or external tools 120, including analytics services 122 or data processing services 124, can also be utilized (e.g., via the communications interface 160) such as to utilize established algorithms or artificial intelligence to complement the system's internal analytics services module 180. In some embodiments, data from patients is anonymized prior to being shared with third party platforms and/or transmitted in a privacy-sensitive secure manner.

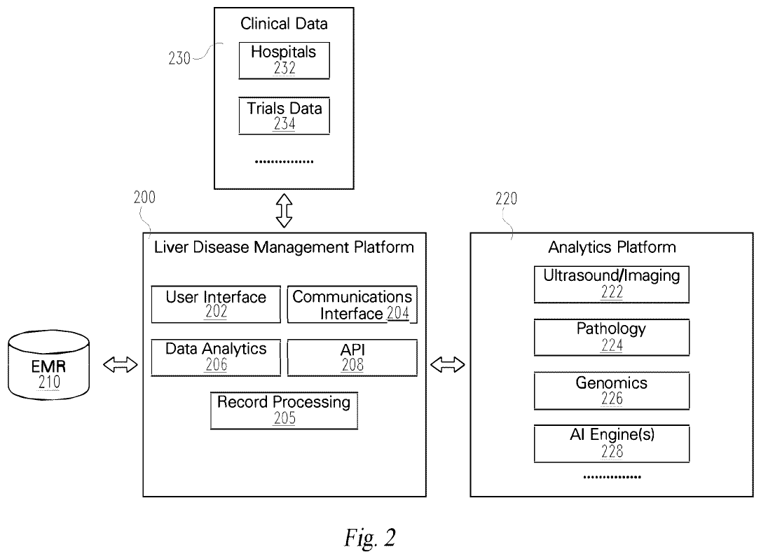

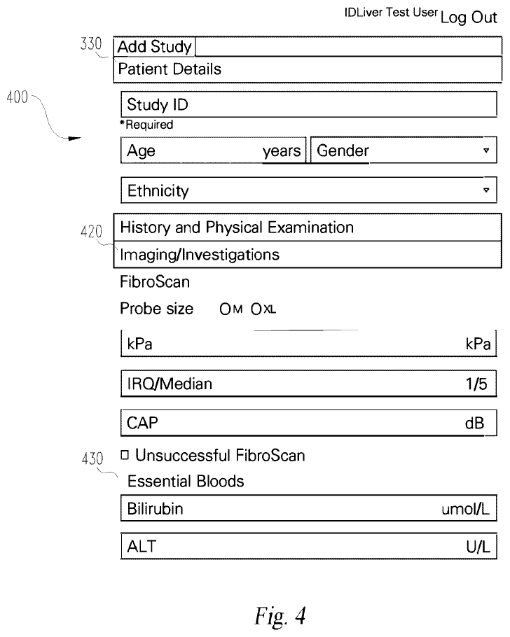

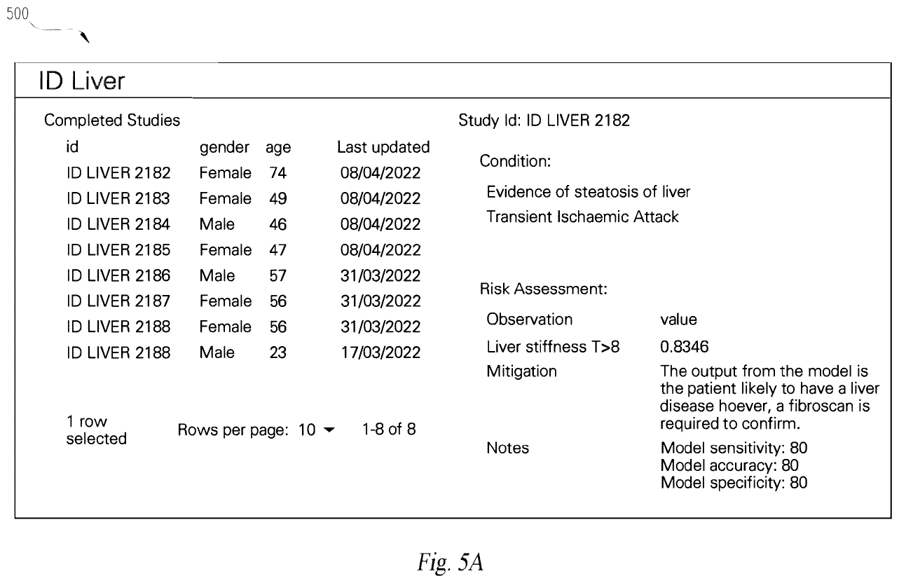

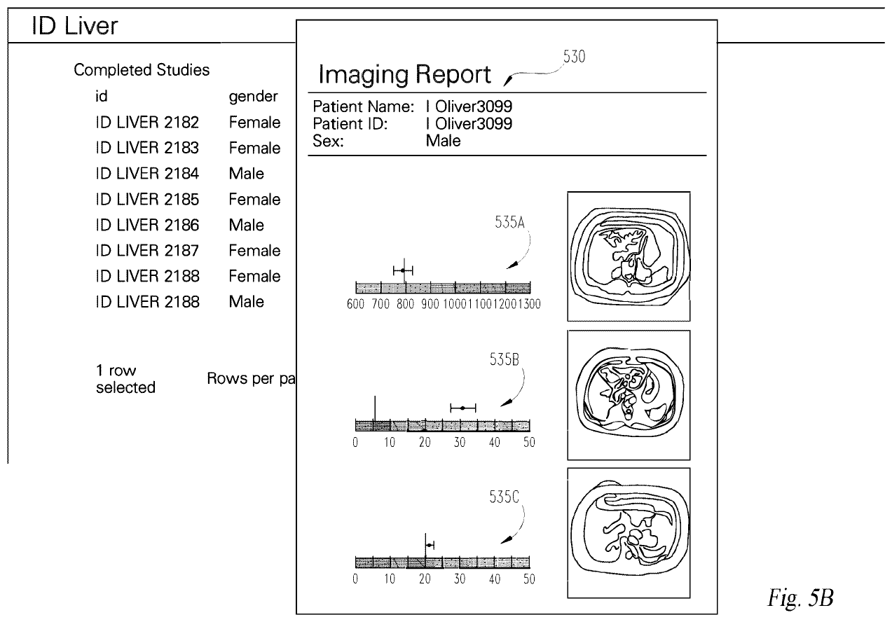

FIG. 2 is an illustrative diagram of a system for managing liver disease according to some embodiments. A liver disease management platform 200 includes a user interface 202 tailored for managing liver disease (e.g., as further shown an described in FIGS. 4, 5A-5B). The interface may be web-based and/or operate as a software program on a remote device (e.g., a laptop, phone, or tablet) through a communications interface 204. The platform 200 is connected to an EMR database 210 (e.g., for a hospital system). A record processing module 205 operates with and structures medical data according to a predefined format for identifying and/or storing data specific to liver conditions (e.g., as described with respect to OCR 145, terminology 146, NER 142, and structuring 148 module of FIG. 1).

A data analytics module 206 is configured to perform data analytics on medical data and, in particular, for identifying liver conditions, prognosis, and potential treatments based on the data. Analytics module 206 may reside in a base medical data platform (e.g., platform 100 of FIG. 1, including analytics services module 180). User interface 202 is configured to obtain and report one or more attributes, conditions, prognosis, and/or recommended treatments relating to liver conditions and/or relative health (e.g., as shown in FIGS. 4 and 5A-5B). User interface 202 or an API interface 208 may be configured to receive analytics queries such as further described with reference to analytics query module 170.

Platform 200 is configured to be connected with analytics platforms 220 for assisting in analyzing data pertaining to liver conditions, including ultrasound/imaging data platform 222, pathology data platform 224, genomics data platform 226, and in providing an artificial intelligence services platform 228. The analytics platforms 220 may reside within a backend (e.g., analytics services module 180) or be connected to external tools such as through API interface 208 to 3rd party tools (e.g., analytics services 122 of FIG. 1). In performing or utilizing analytics, platform 200 may include data 230 such as clinical trials data 234 and hospital/clinic data 232 to augment assessments for identifying liver disease or predicting outcomes in patients and recommending clinical decisions such as described above. Clinical data 230 may be used, for example, to train internal or external machine learning services, or identify potential clinical trials in which patients may engage.

FIG. 3 is an illustrative diagram of a data model for a liver disease management system according to some embodiments. Data records for a patient record 350 may be organized according to behavior/lifestyle records of a patient that may pertain to liver conditions including diet history 370, medication history 380, drug and alcohol history 360, family history 375 (e.g., family history of liver disease), and comorbidities 365 (e.g., cancer, heart disease).

Other records 335 may include specific patient attributes including vital attributes 310 (e.g., height/weight) and diagnostic data including fibroscan tests 315, blood tests 320, ultrasound tests 325, referral indications 330 (e.g., diagnosis). Additional diagnostic data/reports may include imaging reports and data 340 (e.g., MRI, CT, PET) and standardized scores 345 pertaining to liver conditions.

Based on records of a particular patient and assessed in view of similar records of other patients (e.g., using machine learning), assessment(s) 355 of a patient may be generated including diagnosis, risks, and clinical decision recommendations (e.g., further tests, medications, therapies). These assessment(s) 355 may be performed by way of analytics services such as through a backend platform (e.g., analytics services module 180 of FIG. 1) or external analytics platforms (e.g., platforms 220) operated through an interface that protects patient privacy and data security (e.g., API 208 and communications interface 204 of FIG. 2).

FIG. 4 is an illustrative diagram of a user interface 400 for structuring data in a liver disease management system according to some embodiments. A portion of the interface 410 obtains and displays details of a patient that includes details relevant to assessing liver conditions such as described further herein. Details 420 of liver-disease relevant diagnostic tests are also reported/displayed including imaging reports (e.g., a ultrasound/fibroscan, MRI) and of essential blood work at 430. In some embodiments, details displayed in the interface are dynamically obtained from structured or unstructured data (e.g., a pdf of an imaging report) of a patient's record. An operator (e.g., provider) can edit/correct/confirm details shown in the interface such as after a prompt alerting an operator to an update/change in a patient's record.

FIG. 5A is an illustrative diagram of a user interface 500 for structuring and reporting data and analysis in a liver disease management system according to some embodiments. Data obtained from a patient's record is utilized to identify/report liver conditions at 510. The reported conditions may be based on analysis such as performed by integrated analytics services module 180 of FIG. 1 and/or external/independent analytics platforms (e.g., analytics platforms 220 of FIG. 2). A risk assessment 520 is further provided and may also be provided from such analytics platforms.

FIG. 5B is an illustrative diagram of a user interface for structuring and reporting imaging data and analysis in a liver disease management system according to some embodiments. In response to a selection of patient detail or analytical results provided/displayed as part of a user interface (e.g., of FIG. 5A), additional details are provided in an interface 530. Additional details may include different aspects of an imaging study (e.g., imaging layers or transformations) and associated scores 535A, 535B and 535C relating to the identification/assessment of liver conditions or relative liver health. In some embodiments, images obtained in patient records are independently analyzed and scored by the liver disease management system.

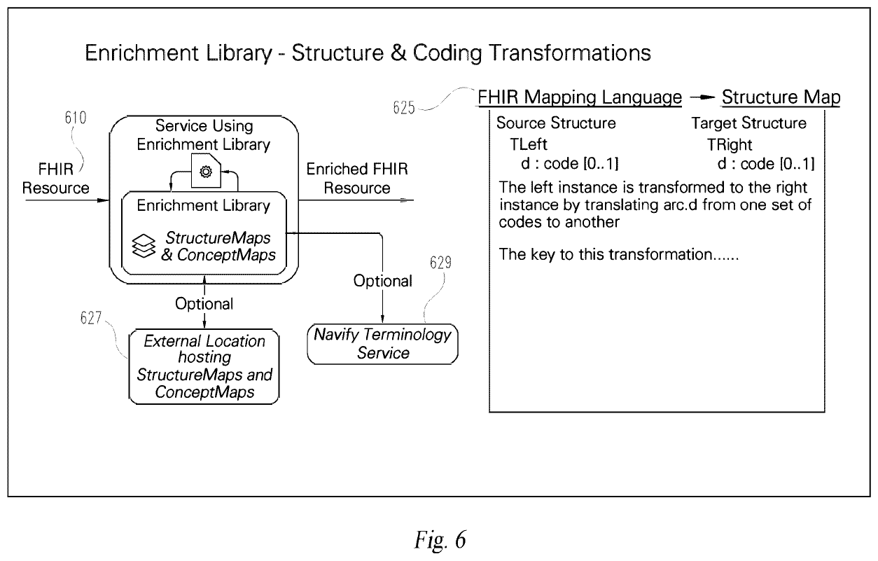

FIG. 6 is an illustrative diagram of a process of structuring and transforming medical data in a liver disease management system according to some embodiments. In some embodiments, data structuring/transformation is performed on patient records in order to transform/structure them to be consistent/harmonized with other aspects of a liver disease management system such as described herein. Data may be received/obtained through a standardized protocol 610 (e.g., FHIR) utilizing a communications interface (e.g., API/Com interfaces 160 of FIG. 1).

An integration service 615 (e.g., an enrichment library 620, integration module 140 of FIG. 1) is utilized to structure and format the data for internal system use. The enrichment library 620 is configured to analyze data (e.g., utilizing OCR module 145 and/or NER module 142) if the data is not already structured in a predetermined way. In some embodiments, the data is transformed into a different form based on a structure map 625 (e.g., translating medical codes/terms from one standard into another).

A customized terminology service 629 and/or external terminology service 627 may be further utilized to transform data according to a custom standard designed to augment identification/analytics for assessing and/or reporting liver conditions and other conditions. Certain types of data may be transformed/integrated based on its type into an applicable format particular to the type as further described herein (e.g., pathology integration module 150, genomics integration module 152, and ultrasound/imaging module 154 of FIG. 1).

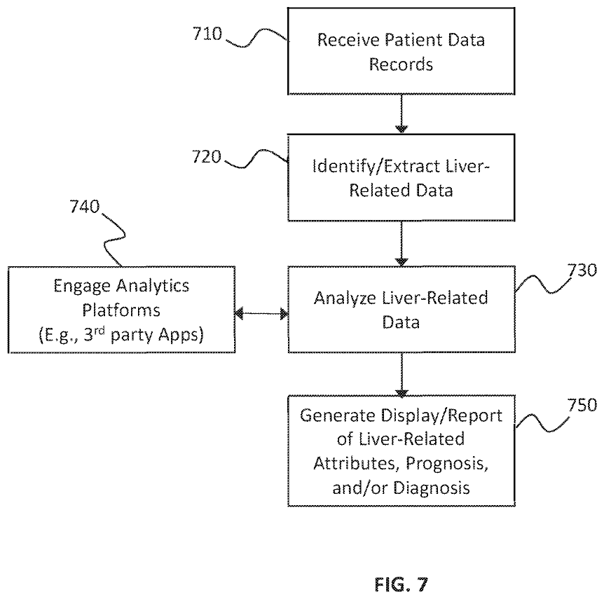

FIG. 7 is an illustrative process flow diagram for generating and reporting liver disease data and clinical analytics according to some embodiments. At block 710, patient data records are received at a system for managing liver disease. The data records may be received from an electronic medical records (EMR) database (e.g., EMR 210 of FIG. 2) such as those used by a hospital system. In some embodiments, the system provides an API by which the system is accessed and records/data interchanged, such as with a hospital's or clinic's EMR platform. The API may be configured to utilize standardized protocols such as FHIR, for example.

In some cases, the records may not be structured or formatted according to a standard for use with a liver disease management system as further described herein (e.g., structure in relation to FIG. 6). At block 720, data from the records is analyzed, based on which data relating to liver disease is identified and extracted. Records may be transformed/structured or new records created based on the identified/extracted data. For example, data from a clinician's report (e. g, from a radiologist/sonographer/hepatologist) that is in a free-form or alternative structure is analyzed (e.g., with an image processor, OCR, and/or NLP processor), from which data pertaining to liver conditions is identified (e.g., identification of a particular biomarker of a liver disease in an image) and structured into a record with particular codes and fields for use within the liver disease management system.

Once in a structured form according to a standard within the liver management system, the structured data may be stored (e.g., in the same or separate EMR database). In some embodiments, the standardized form may be a commonly or widely adopted form readily exchangeable between separate EMR systems (e.g., FHIR adopting standardized codes such as ICD/CPT codes) and/or consistent with a particular EMR system of a healthcare provider. In some embodiments, that data is generated in multiple formats/standards (e.g., original and restructured forms) such as for use with multiple systems.

At block 730, analysis of liver-related data is performed. Such analysis may include predicting diagnosis, risks, and/or determining recommended treatment and/or diagnostic steps. Analysis may include a calculation of a risk/diagnosis based on a predetermined formula of particular data points. For example, a score may be calculated based on particular liver-related diagnostic or imaging result. In some embodiments, the system interfaces with multiple modules each configured for particular data types (e.g., ultrasound/imaging platform 222, pathology platform 224, genomics platform 226 of FIG. 2). In some embodiments, at block 740, more extensive analytics may be utilized to combine analysis including multiple data types such as machine learning tools using multiple biomarkers (e.g., combining genomics, pathology, ultrasound, and chemical analysis) in a patient's record.

The machine learning models or analysis may utilize external or third party tools or data such as from other patients or systems (e.g., clinical data 230 from clinical trials/hospitals) and the models or results may evolve/change over time as more or different data becomes available. An API such as further described herein may be configured to permit the liver disease management system to interface with the external or third party tools. In some embodiments, data from a patient is anonymized prior to transmission to a third party analytical tool.

At block 750, the data and/or analysis pertaining to liver conditions for a patient is reported/presented such as for a clinician or patient. A graphical user interface may be used to report the data and/or analysis (e.g., scores, risk assessments, diagnosis, recommended treatment/diagnostic steps) such as shown in FIG. 5A. In some embodiments, a visualization of clinical images is generated and annotated or complemented with liver-related data and/or analysis (e.g., as shown in FIG. 5B). In some embodiments, presentation of data/analysis pertaining to multiple patients with liver conditions is presented or accessible by way of the interface. Patients with similar history/conditions can be identified and selected for presentation (e.g., for purposes of comparison).

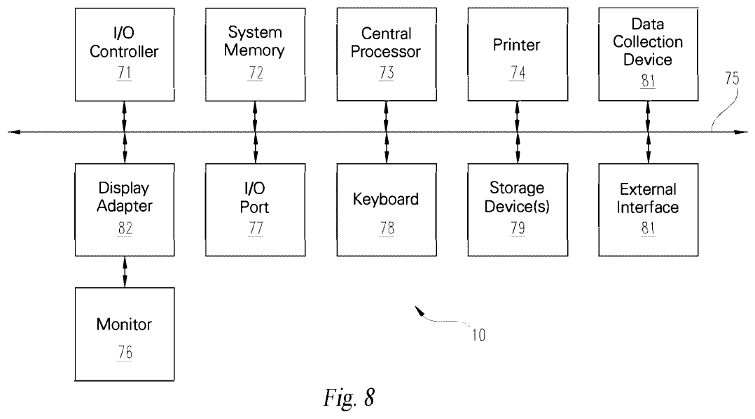

Any of the computer systems mentioned herein, such as for hosting the systems and implementing the processes described for managing liver disease, may utilize any suitable number of subsystems. Examples of such subsystems are shown in FIG. 8 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. In some embodiments, a cloud infrastructure (e.g., Amazon Web Services), a graphical processing unit (GPU), etc., can be used to implement the disclosed techniques.

The subsystems shown in FIG. 8 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Machine learning models utilized herein may include one or more of a Naive Bayes (NB) model, a logistic regression (LR) model, a random forest (RF) model, a support vector machine (SVM) model, an artificial neural network model, a multilayer perceptron (MLP) model, a convolutional neural network (CNN), a Large Language model (LLM), and/or other machine learning or deep leaning models, etc. The machine learning models can be updated/trained using a supervised learning technique, an unsupervised learning technique, etc.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover reference to a “first” or a “second” component does not limit the referenced component to a particular location unless expressly stated.

All patents, patent applications, publications, and descriptions mentioned herein are incorporated by reference in their entirety for all purposes. None is admitted to be prior art.

We claim:

1. A system for managing liver disease, the system comprising:
an electronic medical record (EMR) system comprising medical records of a plurality of patients;
a patient management platform having one or more processors programmed and configured to:
access the EMR system to retrieve the medical records of the plurality of patients;
analyze the medical records to selectively identify data relating to liver condition(s);
interface with one or more data analytics platform(s), the data analytics platform(s) configured to receive and analyze the identified data relating to liver-conditions;
based on the analyzing by the patient management interface and/or the data analytics platform(s), generate a display including specific liver-related patient attributes and conditions, and at least one of a liver-health prognosis or recommended treatment for addressing the liver condition(s) respective to one or more of the plurality patients;
wherein the liver conditions comprise one or more of: cancer, cirrhosis, liver failure, fatty liver disease, steatosis, ischemia, and/or hepatitis.

2. The system of claim 1 wherein the patient management platform comprises a biomarker integration service programmed and configured to communicate biomarker information of the one or more patients to one or more data analytics platforms.

3. The system of claim 1 wherein the data analytics platform(s) comprise a genetics mutation profiler programmed and configured to receive genetics data of the plurality of patients and return the liver-health prognosis or recommended treatment based on analyzing the genetics data.

4. The system of claim 1 wherein accessing the EMR system comprises utilizing application programming interface between the patient management interface and EMR system.

5. The system of claim 1 wherein the patient management platform is programmed and configured to:
receive unstructured or structured data comprising information relating to liver disease;
structuring the data into a predefined format identifying data pertaining to liver conditions; and
storing the structured data in the predefined format within records of the EMR system.

6. The system of claim 5 wherein structuring the data comprises encoding the data into a second format by mapping or transforming the data from a first encoding format into the second format.

7. The system of claim 5 wherein structuring the data comprises encoding the data into at least one or more standards including Fast Healthcare Interoperability Resources (FHIR), SNOMED-CT, LOINC, International Classification of Diseases (ICD), and/or United Code for Units of Measure (UCUM).

8. The system of claim 1 wherein the specific liver-related patient attributes and conditions comprise one or more scores identifying liver health.

9. The system of claim 8 wherein the display presents the one or more scores identifying liver health and presents images of a liver with annotations respectively corresponding to the one or more scores.

10. The system of claim 9 wherein the images comprise one or more of x-ray, ultrasound, and/or MRI images.

11. The system of claim 1 further comprising a communications interface with one or more electronic clinical data repositories having records of clinical data pertaining to a plurality of liver conditions, wherein the interface is configured to provide access to data of the one or more electronic clinical data repositories by the patient management platform and/or the data analytics platform(s) in order to perform data analytics pertaining to liver conditions.

12. The system of claim 11 wherein the patient management platform and/or data analytics platform(s) are configured, using the interface with the one or more electronic clinical data repositories, to train machine learning algorithms for predicting one or more of patient outcomes, clinical decisions, and/or identify therapies for treating liver conditions.

13. A computer implemented method for managing liver disease, the method comprising:
accessing an electronic medical record (EMR) system comprising a plurality of patient records including data relating to liver conditions;
identifying data in the patient records relating to liver condition(s);
interfacing with one or more data analytics platform(s), the data analytics platform(s) configured to receive and analyze the identified data relating to liver-conditions;
based on analyzing by the data analytics platform(s), generating a display including specific liver-related patient attributes and conditions, and at least one of a liver-health prognosis or recommended treatment for addressing the liver condition(s) respective to the one or more patients, wherein the liver conditions comprise one or more of: cancer, cirrhosis, liver failure, fatty liver disease, steatosis, ischemia, and/or hepatitis.

14. The method of claim 13 wherein accessing the EMR system comprises utilizing application programming interface between the patient management interface and EMR system.

15. The method of claim 13 further comprising:
receiving unstructured or structured data comprising information relating to liver disease;
structuring the data into a predefined format identifying data pertaining to liver conditions; and
storing the structured data in the predefined format within records of the electronic medical record system.

16. The method of claim 15 wherein structuring the data comprises encoding the data into a second format by mapping or transforming the data from a first encoding format into the second format.

17. The method of claim 16 wherein the display presents one or more scores identifying liver health and presents images of the liver with annotations respectively corresponding to the one or more scores.

18. The method of claim 17 further comprising accessing one or more electronic clinical data repositories and data analytics platform(s), the one or more electronic clinical data repositories having records of clinical data relating to a plurality of liver conditions, and performing data analytics relating to liver conditions based on data from the one or more clinical data repositories.

19. The method of claim 18 wherein the performing of data analytics comprises applying machine learning algorithms for predicting one or more of patient outcomes, clinical decisions, and/or identify therapies for treating liver conditions, the machine learning algorithms trained with data from the clinical data repositories.

20. A non-transitory computer readable medium with programming instructions for causing one or more processors to perform processing steps comprising:
accessing an electronic medical record (EMR) system comprising a plurality of patient records including data relating to liver conditions;
identifying data in the patient records relating to liver condition(s);
interfacing with one or more data analytics platform(s), the data analytics platform(s) configured to receive and analyze the identified data relating to liver-conditions;
based on analyzing by the data analytics platform(s), generating a display including specific liver-related patient attributes and conditions, and at least one of a liver-health prognosis or recommended treatment for addressing the liver condition(s) respective to the one or more patients, wherein the liver conditions comprise one or more of: cancer, cirrhosis, liver failure, fatty liver disease, steatosis, ischemia, and/or hepatitis.
